SEQUENCE LISTING (2 sequences)

SEQ ID NO: 1 - protein, 9 aa, Artificial Sequence
  Synthetic peptide for conserved motif
LAGLIDADG

SEQ ID NO: 2 - protein, 6 aa, Artificial Sequence
  Synthetic peptide motif
GIYYIG

(19) United States
(12) Patent Application Publication
(10) Pub. No.: US 2013/0071373 A1
(43) Pub. Date: Mar. 21, 2013

(54) METHODS FOR THE DIAGNOSIS AND THERAPY OF RETINITIS PIGMENTOSA

(71) Applicants: Christina Zeitz, Paris (FR); Isabella Audo, Paris (FR); Jose-Alain Sahel, Paris (FR); Shomi Bhattac Harya, Paris (FR)

(72) Inventors: Christina Zeitz, Paris (FR); Isabella Audo, Paris (FR); Jose-Alain Sahel, Paris (FR); Shomi Bhattac Harya, Paris (FR)

(21) Appl. No.: 13/576,688

(22) PCT Filed: Feb. 1, 2011
(86) PCT No.: PCT/EP2011/051378

§ 371 Date: Sep. 18, 2012

(30) Foreign Application Priority Data
Feb. 2, 2010 (EP) 10305108.2

(51) Int. Cl. A61K 38/46; C12Q 1/68

(52) U.S. Cl. CPC C12Q 1/6869; A61K 38/465
USPC 424/94.6; 435/6.11

(57) ABSTRACT

The present invention relates to a method of identifying a subject having or at risk of having or developing a retinitis pigmentosa, comprising detecting in a sample obtained from said subject, the presence of at least one mutation in the rhodopsin (RHO) gene selected from the group consisting of c.263T>C, c.620T>A and c. 1031A>C wherein the presence of said mutation indicates an increased risk of having or being at risk of having or developing the retinitis pigmentosa.

### FIELD OF THE INVENTION

The invention is in the field of retinitis pigmentosa detection, diagnosis, or prognosis. In particular, the invention relates to specific mutations in the human genome and its association with retinitis pigmentosa.

### BACKGROUND OF THE INVENTION

Rod-cone dystrophies, also called retinitis pigmentosa (RP), are a clinically and genetically heterogeneous group of inherited retinal disorders primarily affecting rods with secondary cone degeneration. Most patients affected with RP initially complain of night blindness due to rod dysfunction followed by progressive visual field constriction, abnormal color vision and which can eventually lead to loss of central vision due to secondary cone degeneration leading to severe visual handicap and blindness. It is the most common inherited form of severe retinal degeneration, with a frequency of about 1 in 4000 births and more than 1 million affected individuals over the world. The mode of inheritance can be X-linked (5-15%) autosomal dominant (30-40%) or autosomal recessive (50-60%) The remaining patients represent isolated cases of which the inheritance trait can not be established.

To date, 20 autosomal dominant RP (adRP) genes have been reported (http://www.sph.uth.tmc.edu/Retnet/). One of the major genes underlying this disorder is rhodopsin (RHO) coding for the light absorbing molecule that initiates the signal transmission cascade in rod photoreceptors. According to the literature, RHO mutation prevalence ranges from 0 to 50% cases of adRP in cohorts from various geographical origins, with higher numbers reported in the United States.

The genetic and phenotypic heterogeneity is not only found in RP in general but also specifically reflected in adRP with RHO mutations: Over 120 mutations have been identified in different sites of the gene including specific hot spots (http://www.sph.uth.tmc.edu/Retnet/, http://www.hgmd.cf.ac.uk/ac/all.php, http://www.retina-international.org/sci-news/rhomut.htm).

Certain mutations in RHO lead to diffuse rod-cone dysfunction whereas other cases are implicated in a more restricted disease that may predominate in the inferior part of the retina such as in sector RP. Phenotypic classifications have been proposed to reflect this variability. In particular, Cideciyan and co-workers have distinguished two classes of disease expression with allele-specificity: class A mutants show severely generalized abnormal rod function early in life with a constant rate of cone disease progression across the retina with time. Class B mutants show more restricted disease and absent or late-onset night blindness.

Other classifications have been proposed based on the underlying pathogenic mechanism involved in adRP due to RHO mutations. Mendes and co-workers classified the different types of mutations in 6 groups. Class I refers exclusively to rhodopsin mutations that fold correctly but are not transported to the outer segment. Class II, refers to mutations that misfold, are retained in the endopasmic reticulum (ER) and cannot easily reconstitute with 11-cis-retinal. Class III refers to mutations that affect endocytosis. Class IV mutations do not affect folding per se but might affect rhodopsin stability and posttranslational modification. Similarly, Class V mutations have no obvious folding defect but show an increased activation rate for transducin. Mutants that appear to fold correctly but lead to the constitutive activation of opsin in the absence of the chromophore and in the dark constitute Class VI. Other mutations with unclear biochemical or cellular defect, or uninvestigated defect were not classified.

Nonetheless, up to this point, no single biomarker is sufficiently specific to provide adequate clinical utility for the diagnosis of retinitis pigmentosa in an individual subject. Therefore, there is a need for identifying other mutations that provide a more accurate diagnosis/prognosis of retinitis pigmentosa. Thus, the invention aims to provide a novel method for the diagnosis/prognosis of retinitis pigmentosa.

### SUMMARY OF THE INVENTION

The present invention relates to a method of identifying a subject having or at risk of having or developing a retinitis pigmentosa, comprising detecting in a sample obtained from said subject, the presence of at least one mutation in the rhodopsin (RHO) gene selected from the group consisting of c.263T>C, c.620T>A and c.1031A>C wherein the presence of said mutation indicates an increased risk of having or being at risk of having or developing the retinitis pigmentosa.

### DETAILED DESCRIPTION OF THE INVENTION

### Definitions

Throughout the specification, several terms are employed and are defined in the following paragraphs.

Throughout the specification, several terms are employed and are defined in the following paragraphs.

A “coding sequence” or a sequence “encoding” an expression product, such as a RNA, polypeptide, protein, or enzyme, is a nucleotide sequence that, when expressed, results in the production of that RNA, polypeptide, protein, or enzyme, i.e., the nucleotide sequence encodes an amino acid sequence for that polypeptide, protein or enzyme. A coding sequence for a protein may include a start codon (usually ATG) and a stop codon.

The term “gene” means a DNA sequence that codes for or corresponds to a particular sequence of amino acids which comprise all or part of one or more proteins or enzymes, and may or may not include regulatory DNA sequences, such as promoter sequences, which determine for example the conditions under which the gene is expressed. Some genes, which are not structural genes, may be transcribed from DNA to RNA, but are not translated into an amino acid sequence. Other genes may function as regulators of structural genes or as regulators of DNA transcription. In particular, the term gene may be intended for the genomic sequence encoding a protein, i.e. a sequence comprising regulator, promoter, intron and exon sequences.

As used herein, the term “rhodopsin gene” or “RHO” denotes the rhodospin gene of any species, especially human, but also other mammals or vertebrates to which the methods of the invention can apply. The human rhodopsin gene is located at 3q21-q24 1 and is composed of 5 exons. The transcript is 2768 by long (accession number NM_{—}000539). The encoded protein rhodopsin is 348 amino-acids long (accession number NP_{—}000530). We have chosen to number the A of the start codon (ATG) of the cDNA sequence of rhodopsin (Genbank accession numbers NM_{—}000539) as nucleotide 1.

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength (see Sambrook et al., 1989).

The conditions of temperature and ionic strength determine the “stringency” of the hybridization. For preliminary screening for homologous nucleic acids, low stringency hybridization conditions, corresponding to a Tm (melting temperature) of 55° C., can be used, e.g., 5× SSC, 0.1% SDS, 0.25% milk, and no formamide; or 30% formamide, 5× SSC, 0.5% SDS). Moderate stringency hybridization conditions correspond to a higher Tm, e.g., 40% formamide, with 5× or 6× SCC. High stringency hybridization conditions correspond to the highest Tm, e.g., 50% formamide, 5× or 6× SCC. SCC is a 0.15 M NaCl, 0.015 M Na-citrate. Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tm for hybrids of nucleic acids having those sequences. The relative stability (corresponding to higher Tm) of nucleic acid hybridizations decreases in the following order: RNA:RNA, DNA:RNA, DNA:DNA. For hybrids of greater than 100 nucleotides in length, equations for calculating Tm have been derived (see Sambrook et al., 1989, 9.50-9.51). For hybridization with shorter nucleic acids, i.e., oligonucleotides, the position of mismatches becomes more important, and the length of the oligonucleotide determines its specificity (see Sambrook et al., 1989 11.7-11.8). A minimum length for a hybridizable nucleic acid is at least about 10 nucleotides, preferably at least about 15 nucleotides, and more preferably the length is at least about 20 nucleotides.

In a specific embodiment, the term “standard hybridization conditions” refers to a Tm of 55° C., and utilizes conditions as set forth above. In a preferred embodiment, the Tm is 60° C. In a more preferred embodiment, the Tm is 65° C. In a specific embodiment, “high stringency” refers to hybridization and/or washing conditions at 68° C. in 0.2 X SSC, at 42° C. in 50% formamide, 4 X SSC, or under conditions that afford levels of hybridization equivalent to those observed under either of these two conditions.

As used herein, an amplification primer is an oligonucleotide for amplification of a target sequence by extension of the oligonucleotide after hybridization to the target sequence or by ligation of multiple oligonucleotides which are adjacent when hybridized to the target sequence. At least a portion of the amplification primer hybridizes to the target. This portion is referred to as the target binding sequence and it determines the target-specificity of the primer. In addition to the target binding sequence, certain amplification methods require specialized non-target binding sequences in the amplification primer. These specialized sequences are necessary for the amplification reaction to proceed and typically serve to append the specialized sequence to the target. For example, the amplification primers used in Strand Displacement Amplification (SDA) include a restriction endonuclease recognition site 5′ to the target binding sequence (U.S. Pat. No. 5,455,166 and U.S. Pat. No. 5,270,184). Nucleic Acid Based Amplification (NASBA), self-sustaining sequence replication (3SR) and transcription based amplification primers require an RNA polymerase promoter linked to the target binding sequence of the primer. Linking such specialized sequences to a target binding sequence for use in a selected amplification reaction is routine in the art. In contrast, amplification methods such as PCR which do not require specialized sequences at the ends of the target, generally employ amplification primers consisting of only target binding sequence.

As used herein, the terms “primer” and “probe” refer to the function of the oligonucleotide. A primer is typically extended by polymerase or ligation following hybridization to the target but a probe typically is not. A hybridized oligonucleotide may function as a probe if it is used to capture or detect a target sequence, and the same oligonucleotide may function as a primer when it is employed as a target binding sequence in an amplification primer. It will therefore be appreciated that any of the target binding sequences disclosed herein for amplification, detection or quantisation of rhodopsin may be used either as hybridization probes or as target binding sequences in primers for detection or amplification, optionally linked to a specialized sequence required by the selected amplification reaction or to facilitate detection.

The terms “mutant” and “mutation” mean any detectable change in genetic material, e.g. DNA, RNA, cDNA, or any process, mechanism, or result of such a change. This includes gene mutations, in which the structure (e.g. DNA sequence) of a gene is altered, any gene or DNA arising from any mutation process, and any expression product (e.g. protein or enzyme) expressed by a modified gene or DNA sequence. Generally a mutation is identified in a subject by comparing the sequence of a nucleic acid or polypeptide expressed by said subject with the corresponding nucleic acid or polypeptide expressed in a control population. A mutation in the genetic material may also be “silent”, i.e. the mutation does not result in an alteration of the amino acid sequence of the expression product.

In the context of the instant application, mutations identified in rhodopsin gene are designated pursuant to the nomenclature of Dunnen and Antonarakis (2000). As defined by Dunnen and Antonarakis at the nucleic acid level, substitutions are designated by “>”, e.g. “263T>C” denotes that at nucleotide 263 of the reference sequence a T is changed to a C.

In the context of the invention, the term “treating” or “treatment”, as used herein, means reversing, alleviating, inhibiting the progress of, or preventing the disorder or condition to which such term applies, or one or more symptoms of such disorder or condition. A “therapeutically effective amount” is intended for a minimal amount of active agent (e.g., rhodopsin encoding polynucleotide) which is necessary to impart therapeutic benefit to a subject. For example, a “therapeutically effective amount” to a mammal is such an amount which induces, ameliorates or otherwise causes an improvement in the pathological symptoms, disease progression or physiological conditions associated with or resistance to succumbing to a disorder.

The term “biological sample” means any biological sample derived from a subject. Examples of such samples include fluids, tissues, cell samples, organs, biopsies, etc. Preferred biological samples are a cell or tissue sample. Preferred biological samples are whole blood, serum, plasma or urine. Typically, in the case where the subject is a foetus, the sample may be an amniosynthesis sample.

A “subject” in the context of the present invention is preferably a mammal. The mammal can be a human, non-human primate, mouse, rat, dog, cat, horse, or cow, but are not limited to these examples. Mammals other than humans can be advantageously used as subjects that represent animal models of retinitis pigmentosa. A subject can be male or female. A subject can also be one who has not been previously diagnosed as having retinitis pigmentosa. For example, a subject can be one who exhibits one or more risk factors for retinitis pigmentosa, or a subject who does not exhibit retinitis pigmentosa risk factors, or a subject who is asymptomatic for retinitis pigmentosa. A subject can also be one who is at risk of developing retinitis pigmentosa.

### Diagnostic Methods of the Invention

The present invention relates to a method of identifying a subject having or at risk of having or developing a retinitis pigmentosa, comprising detecting in a sample obtained from said subject, the presence of at least one mutation in the rhodopsin (RHO) gene selected from the group consisting of c.263T>C, c.620T>A and c.1031A>C wherein the presence of said mutation indicates an increased risk of having or being at risk of having or developing a retinitis pigmentosa.

In one embodiment of the invention, the subject having or being at risk of having or developing a retinitis pigmentosa may be a substantially healthy subject, which means that the subject has not been previously diagnosed or identified as having or suffering from a retinitis pigmentosa.

In another embodiment, said subject may also be one that is asymptomatic for retinitis pigmentosa. As used herein, an “asymptomatic” subject refers to a subject that does not exhibit the traditional symptoms of retinitis pigmentosa.

In another embodiment of the invention, said subject may be one that is at risk of having or developing retinitis pigmentosa, as defined by clinical indicia such as for example: age, gender, family history of retinitis pigmentosa, night blindness, precursors of AMD on fundus examination.

In one embodiment of the invention, the subject is a mammal, preferably a human.

RHO mutations may be detected in a RNA or DNA sample, preferably after amplification. For instance, the isolated RNA may be subjected to coupled reverse transcription and amplification, such as reverse transcription and amplification by polymerase chain reaction (RT-PCR), using specific oligonucleotide primers that are specific for a mutated site or that enable amplification of a region containing the mutated site. According to a first alternative, conditions for primer annealing may be chosen to ensure specific reverse transcription (where appropriate) and amplification; so that the appearance of an amplification product be a diagnostic of the presence of a particular RHO mutation. Otherwise, RNA may be reverse-transcribed and amplified, or DNA may be amplified, after which a mutated site may be detected in the amplified sequence by hybridization with a suitable probe or by direct sequencing, or any other appropriate method known in the art. For instance, a cDNA obtained from RNA may be cloned and sequenced to identify a mutation in RHO sequence.

Actually numerous strategies for genotype analysis are available (Antonarakis et al., 1989; Cooper et al., 1991; Grompe, 1993). Briefly, the nucleic acid molecule may be tested for the presence or absence of a restriction site. When a base substitution mutation creates or abolishes the recognition site of a restriction enzyme, this allows a simple direct PCR test for the mutation. Further strategies include, but are not limited to, direct sequencing, restriction fragment length polymorphism (RFLP) analysis; hybridization with allele-specific oligonucleotides (ASO) that are short synthetic probes which hybridize only to a perfectly matched sequence under suitably stringent hybridization conditions; allele-specific PCR; PCR using mutagenic primers; ligase-PCR, HOT cleavage; denaturing gradient gel electrophoresis (DGGE), temperature denaturing gradient gel electrophoresis (TGGE), single-stranded conformational polymorphism (SSCP) and denaturing high performance liquid chromatography (Kuklin et al., 1997). Direct sequencing may be accomplished by any method, including without limitation chemical sequencing, using the Maxam-Gilbert method; by enzymatic sequencing, using the Sanger method; mass spectrometry sequencing; sequencing using a chip-based technology; and real-time quantitative PCR. Preferably, DNA from a subject is first subjected to amplification by polymerase chain reaction (PCR) using specific amplification primers. However several other methods are available, allowing DNA to be studied independently of PCR, such as the rolling circle amplification (RCA), the InvaderTMassay, or oligonucleotide ligation assay (OLA). OLA may be used for revealing base substitution mutations. According to this method, two oligonucleotides are constructed that hybridize to adjacent sequences in the target nucleic acid, with the join sited at the position of the mutation. DNA ligase will covalently join the two oligonucleotides only if they are perfectly hybridized.

Therefore, useful nucleic acid molecules, in particular oligonucleotide probes or primers, according to the present invention include those which specifically hybridize the regions where the mutations are located. Oligonucleotide probes or primers may contain at least 10, 15, 20 or 30 nucleotides. Their length may be shorter than 400, 300, 200 or 100 nucleotides.

According to a further embodiment said mutation in the RHO gene may be detected at the protein level. For example, mutation c.263T>C in RHO gene leads to a p.Leu88Pro mutation at protein level. Mutation c.620T>A in RHO gene leads to p.Met207Lys mutation at protein level. Finally, mutation c.1031A>C in RHO gene leads to mutation p.Gln344Pro at protein level.

Said mutation may be detected according to any appropriate method known in the art. In particular a sample, such as a tissue biopsy, obtained from a subject may be contacted with antibodies specific of the mutated form of RHO, i.e. antibodies that are capable of distinguishing between a mutated form of RHO and the wild-type protein (or any other protein), to determine the presence or absence of a RHO specified by the antibody.

Antibodies that specifically recognize a mutated RHO also make part of the invention. The antibodies are specific of mutated RHO that is to say they do not cross-react with the wild-type RHO.

The antibodies of the present invention may be monoclonal or polyclonal antibodies, single chain or double chain, chimeric antibodies, humanized antibodies, or portions of an immunoglobulin molecule, including those portions known in the art as antigen binding fragments Fab, Fab′, F(ab′)2 and F(v). They can also be immunoconjugated, e.g. with a toxin, or labelled antibodies.

Whereas polyclonal antibodies may be used, monoclonal antibodies are preferred for they are more reproducible in the long run.

Procedures for raising “polyclonal antibodies” are also well known. Polyclonal antibodies can be obtained from serum of an animal immunized against the appropriate antigen, which may be produced by genetic engineering for example according to standard methods well-known by one skilled in the art. Typically, such antibodies can be raised by administering mutated RHO subcutaneously to New Zealand white rabbits which have first been bled to obtain pre-immune serum. The antigens can be injected at a total volume of 100 μl per site at six different sites. Each injected material may contain adjuvants with or without pulverized acrylamide gel containing the protein or polypeptide after SDS-polyacrylamide gel electrophoresis. The rabbits are then bled two weeks after the first injection and periodically boosted with the same antigen three times every six weeks. A sample of serum is then collected 10 days after each boost. Polyclonal antibodies are then recovered from the serum by affinity chromatography using the corresponding antigen to capture the antibody. This and other procedures for raising polyclonal antibodies are disclosed by Harlow et al. (1988) which is hereby incorporated in the references.

A “monoclonal antibody” in its various grammatical forms refers to a population of antibody molecules that contains only one species of antibody combining site capable of immunoreacting with a particular epitope. A monoclonal antibody thus typically displays a single binding affinity for any epitope with which it immunoreacts. A monoclonal antibody may therefore contain an antibody molecule having a plurality of antibody combining sites, each immunospecific for a different epitope, e.g. a bispecific monoclonal antibody. Although historically a monoclonal antibody was produced by immortalization of a clonally pure immunoglobulin secreting cell line, a monoclonally pure population of antibody molecules can also be prepared by the methods of the present invention.

Laboratory methods for preparing monoclonal antibodies are well known in the art (see, for example, Harlow et al., 1988). Monoclonal antibodies (mAbs) may be prepared by immunizing purified mutated RHO into a mammal, e.g. a mouse, rat, human and the like mammals. The antibody-producing cells in the immunized mammal are isolated and fused with myeloma or heteromyeloma cells to produce hybrid cells (hybridoma). The hybridoma cells producing the monoclonal antibodies are utilized as a source of the desired monoclonal antibody. This standard method of hybridoma culture is described in Kohler and Milstein (1975).

While mAbs can be produced by hybridoma culture the invention is not to be so limited. Also contemplated is the use of mAbs produced by an expressing nucleic acid cloned from a hybridoma of this invention. That is, the nucleic acid expressing the molecules secreted by a hybridoma of this invention can be transferred into another cell line to produce a transformant. The transformant is genotypically distinct from the original hybridoma but is also capable of producing antibody molecules of this invention, including immunologically active fragments of whole antibody molecules, corresponding to those secreted by the hybridoma. See, for example, U.S. Pat. No. 4,642,334 to Reading; PCT Publication No.; European Patent Publications No. 0239400 to Winter et al. and No. 0125023 to Cabilly et al.

Antibody generation techniques not involving immunisation are also contemplated such as for example using phage display technology to examine naive libraries (from non-immunised animals); see Barbas et al. (1992), and Waterhouse et al. (1993). Antibodies raised against mutated RHO may be cross reactive with wild-type RHO.

Accordingly a selection of antibodies specific for mutated RHO is required. This may be achieved by depleting the pool of antibodies from those that are reactive with the wild-type RHO, for instance by submitting the raised antibodies to an affinity chromatography against wild-type RHO.

Alternatively, binding agents other than antibodies may be used for the purpose of the invention. These may be for instance aptamers, which are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. and Gold L., 1990. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S.D., 1999. Peptide aptamers consists of a conformationally constrained antibody variable region displayed by a platform protein, such as E. coli Thioredoxin A that are selected from combinatorial libraries by two hybrid methods (Colas et al., 1996).

Probe, primers, aptamers or antibodies of the invention may be labelled with a detectable molecule or substance, such as a fluorescent molecule, a radioactive molecule or any others labels known in the art. Labels are known in the art that generally provide (either directly or indirectly) a signal.

The term “labelled”, with regard to the probe, primers, aptamers or antibodies of the invention, is intended to encompass direct labelling of the probe, primers, aptamers or antibodies of the invention by coupling (i.e., physically linking) a detectable substance to the the probe, primers, aptamers or antibodies of the invention, as well as indirect labeling of the probe, primers, aptamers or antibodies of the invention by reactivity with another reagent that is directly labeled. Other examples of detectable substances include but are not limited to radioactive agents or a fluorophore (e.g. fluorescein isothiocyanate (FITC) or phycoerythrin (PE) or Indocyanine (Cy5)). Examples of indirect labeling include detection of a primary antibody using a fluorescently labeled secondary antibody and end-labeling of a DNA probe with biotin such that it can be detected with fluorescently labeled streptavidin. An antibody or aptamer of the invention may be labelled with a radioactive molecule by any method known in the art. For example radioactive molecules include but are not limited radioactive atom for scintigraphic studies such as I123, I124, In111, Re186, Re188.

### Kits of the Invention

According to another aspect of the invention, the RHO mutation is detected by contacting the DNA of the subject with a nucleic acid probe, which is optionally labelled.

Primers may also be useful to amplify or sequence the portion of the RHO gene containing the mutated positions of interest.

Such probes or primers are nucleic acids that are capable of specifically hybridizing with a portion of the RHO gene sequence containing the mutated positions of interest. That means that they are sequences that hybridize with the portion mutated RHO nucleic acid sequence to which they relate under conditions of high stringency.

The present invention further provides kits suitable for determining at least one of the mutations of the RHO gene.

The kits may include the following components:
(i) a probe, usually made of DNA, and that may be pre-labelled. Alternatively, the probe may be unlabelled and the ingredients for labelling may be included in the kit in separate containers; and
(ii) hybridization reagents: the kit may also contain other suitably packaged reagents and materials needed for the particular hybridization protocol, including solid-phase matrices, if applicable, and standards.

In another embodiment, the kits may include:
(i) sequence determination or amplification primers: sequencing primers may be pre-labelled or may contain an affinity purification or attachment moiety; and
(ii) sequence determination or amplification reagents: the kit may also contain other suitably packaged reagents and materials needed for the particular sequencing amplification protocol. In one preferred embodiment, the kit comprises a panel of sequencing or amplification primers, whose sequences correspond to sequences adjacent to at least one of the polymorphic positions, as well as a means for detecting the presence of each polymorphic sequence.

In a particular embodiment, it is provided a kit which comprises a pair of nucleotide primers specific for amplifying all or part of the RHO gene comprising at least one of mutations that are identified herein.

Alternatively, the kit of the invention may comprise a labelled compound or agent capable of detecting the mutated polypeptide of the invention (e.g., an antibody or aptamers as described above which binds the polypeptide). For example, the kit may comprise (1) a first antibody (e.g., attached to a solid support) which binds to a polypeptide comprising a mutation of the invention; and, optionally, (2) a second, different antibody which binds to either the polypeptide or the first antibody and is conjugated to a detectable agent.

The kit can also comprise, e.g., a buffering agent, a preservative, or a protein stabilizing agent. The kit can also comprise components necessary for detecting the detectable agent (e.g., an enzyme or a substrate). The kit can also contain a control sample or a series of control samples which can be assayed and compared to the test sample contained. Each component of the kit is usually enclosed within an individual container and all of the various containers are within a single package along with instructions for observing whether the tested subject is suffering from or is at risk of developing a retinitis pigmentosa.

### Therapeutic Methods of the Invention

In a further object, the invention relates to use, methods and pharmaceutical compositions for treating or preventing a retinitis pigmentosa. Gene therapy is a particularly convenient way to treat a retinitis pigmentosa as it enables the provision of a constant supply of polypeptide or correction of the defective gene, for example as discussed below.

Gene therapy may be carried out by means of supplementation of cells lacking a functional RHO polypeptide with a wild type RHO gene product. Production of a suitable gene product may be achieved using recombinant techniques. For example, a suitable vector may be inserted into a host cell and expressed in that cell.

Thus the invention further relates to a method for treating or preventing a retinitis pigmentosa which comprises the step of administering a subject in need thereof with a RHO polynucleotide, i.e. a nucleic acid sequence that encodes a wild-type RHO, so that RHO is expressed in vivo by the cells of the subject that have been transfected with said polynucleotide. Accordingly, said method leads to an overexpression of wild-type RHO which compensates expression of defective mutated RHO.

The invention also relates to the use of a RHO polynucleotide for the manufacture of medicament intended for the treatment of a retinitis pigmentosa.

Said RHO polynucleotide is administered in a therapeutically effective amount.

Preferably the RHO polynucleotide sequence according to the invention is associated with elements that enable for regulation of its expression, such as a promoter sequence.

Such a nucleic acid may be in the form of a vector. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, expression vectors, are capable of directing the expression of genes to which they are operably linked. In general, expression vectors of utility in recombinant DNA techniques are often in the form of plasmids (vectors). However, the invention is intended to include such other forms of expression vectors, such as viral vectors (e.g., replication defective retroviruses, adenoviruses and adeno-associated viruses (AAV)), which serve equivalent functions.

In a preferred embodiment, the expression vector is an AAV vector. Adeno-associated viral (AAV) vectors have become powerful gene delivery tools for the treatment of retinal degeneration. AAV vectors possess a number of features that render them ideally suited for retinal gene therapy, including a lack of pathogenicity, minimal immunogenicity, and the ability to transduce postmitotic cells in a stable and efficient manner. In the sheltered environment of the retina, AAV vectors are able to maintain high levels of transgene expression in the retinal pigmented epithelium (RPE), photoreceptors, or ganglion cells for long periods of time after a single treatment. Each cell type can be specifically targeted by choosing the appropriate combination of AAV serotype, promoter, and intraocular injection site (Dinculescu et al., Hum Gene Ther. 2005 June; 16(6):649-63).

The RHO polynucleotide may be introduced into a target cell by means of any procedure known for the delivery of nucleic acids to the nucleus of cells, ex vivo, on cells in culture or removed from an animal or a patient, or in vivo.

Ex vivo introduction may be performed by any standard method well known by one skilled in the art, e.g. transfection, electroporation, microinjection, transduction, cell fusion, DEAE dextran, calcium phosphate precipitation, or use of a gene gun.

The RHO polynucleotide can also be introduced ex vivo or in vivo by lipofection. In certain embodiments, the use of liposomes and/or nanoparticles is contemplated for the introduction of the donor nucleic acid targeting system into host cells.

Nanocapsules can generally entrap compounds in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use in the present invention, and such particles may be are easily made.

Liposomes are formed from phospholipids that are dispersed in an aqueous medium and spontaneously form multilamellar concentric bilayer vesicles (also termed multilamellar vesicles (MLVs)). MLVs generally have diameters of from 25 nm to 4 μm. Sonication of MLVs results in the formation of small unilamellar vesicles (SUVs) with diameters in the range of 200 to 500 Å, containing an aqueous solution in the core.

Synthetic cationic lipids designed to limit the difficulties and dangers encountered with liposome mediated transfection can be used to prepare liposomes for in vivo transfection of a gene encoding a marker. The use of cationic lipids may promote encapsulation of negatively charged nucleic acids, and also promote fusion with negatively charged cell membranes (Felgner et al., 1989).

Alternatively, one of the simplest and the safest way to deliver RHO polynucleotide across cell membranes in vivo may involve the direct application of high concentration free or naked polynucleotides (typically mRNA or DNA). By “naked DNA (or RNA)” is meant a DNA (RNA) molecule which has not been previously complexed with other chemical moieties. Naked DNA uptake by animal cells may be increased by administering the cells simultaneously with excipients and the nucleic acid. Such excipients are reagents that enhance or increase penetration of the DNA across cellular membranes and thus delivery to the cells delivery of the therapeutic agent. Various excipients have been described in the art, such as surfactants, e.g. a surfactant selected form the group consisting of Triton X-100, sodium dodecyl sulfate, Tween 20, and Tween 80; bacterial toxins, for instance streptolysin O, cholera toxin, and recombinant modified labile toxin of E coli; and polysaccharides, such as glucose, sucrose, fructose, or maltose, for instance, which act by disrupting the osmotic pressure in the vicinity of the cell membrane. Other methods have been described to enhance delivery of free polynucleotides, such as blocking of polynucleotide inactivation via endo- or exonucleolytic cleavage by both extra- and intracellular nucleases.

Alternatively, the invention also provides a method for treating or preventing a retinitis pigmentosa which comprises the step of administering a subject in need thereof with a wild-type RHO.

Knowing the amino acid sequence of the desired sequence, one skilled in the art can readily produce said polypeptides, by standard techniques for production of polypeptides. For instance, they can be synthesized using well-known solid phase method, preferably using a commercially available peptide synthesis apparatus (such as that made by Applied Biosystems, Foster City, Calif.) and following the manufacturer's instructions.

Alternatively, the polypeptides of the invention can be synthesized by recombinant DNA techniques as is now well-known in the art. For example, these fragments can be obtained as DNA expression products after incorporation of DNA sequences encoding the desired (poly)peptide into expression vectors and introduction of such vectors into suitable eukaryotic or prokaryotic hosts that will express the desired polypeptide, from which they can be later isolated using well-known techniques.

Polypeptides of the invention can be use in an isolated (e.g., purified) form or contained in a vector, such as a membrane or lipid vesicle (e.g. a liposome).

A further aspect of the invention relates to a meganuclease for use in the treatment of retinitis pigmentosa wherein said meganuclease cleaves a DNA target sequence in a RHO gene locus which comprises a mutation selected from the group consisting of c.263T>C, c.620T>A and c.1031A>C.

In the present application, by “meganuclease” is intended a double-stranded endonuclease having a large polynucleotide recognition site, at least 12 bp, preferably from 12 by to 60 bp. Said meganuclease is also called rare-cutting or very rare-cutting endonuclease. Said meganuclease is either monomeric or dimeric. It includes any natural meganuclease such as a homing endonuclease, but also any artificial or man-made meganuclease endowed with such high specificity, either derived from homing endonucleases of group I introns and inteins, or other proteins such as Zinc-Finger proteins or group II intron proteins, or compounds such as nucleic acid fused with chemical compounds. In particular, artificial meganucleases include the so-called “custom-made meganuclease” which is a meganuclease derived from any initial meganuclease, either natural or not, presenting a recognition and cleavage site different from the site of the initial one; zinc-finger nucleases may also be considered as custom-made meganucleases. By “different” is intended that the custom-made meganuclease cleaves the novel site with an efficacy at least 10 fold more than the natural meganuclease, preferably at least 50 fold, more preferably at least 100 fold. “Natural” refers to the fact that an object can be found in nature. For example, a meganuclease that is present in an organism, that can be isolated from a source in nature and which has not been intentionally modified by man in the laboratory is natural.

In particular embodiment, said meganuclease is selected from the group consisting of a homing endonuclease, a zinc-finger nuclease or a custom-made meganuclease.

Typically, homing endonucleases are described in Chevalier et al. (Chevalier and Stoddard, 2001, N.A.R., 29, 3757-74).

Meganuclease based on Zinc-Finger domains have the structure described by Smith et al. (Smith et al, 2000, N.A.R, 28, 3361-9; Smith et al., 1999, Nucleic Acids Res., 27, 274-281).

Custom-made meganuclease is defined as a meganuclease able to cleave a targeted DNA sequence. This definition includes any meganuclease variant produced by a method comprising the steps of preparing a library of meganuclease variants and isolating, by selection and/or screening, the variants able to cleave the targeted DNA sequence. Said custom-made meganuclease which is derived from any initial meganuclease by introduction of diversity, presents a recognition and cleavage site different from the site of the initial one.

The diversity could be introduced in the meganuclease by any method available for the man skilled in the art. Preferably, the diversity is introduced by targeted mutagenesis (i.e. cassette mutagenesis, oligonucleotide directed codon mutagenesis, targeted random mutagenesis), by random mutagenesis (i.e. mutator strains, Neurospora crassa system (U.S. Pat. No. 6,232,112; WO01/70946, error-prone PCR), by DNA shuffling, by directed mutation or a combination of these technologies (See Current Protocols in Molecular Biology, Chapter 8 “Mutagenesis in cloned DNA”, Eds Ausubel et al., John Wiley and Sons). The meganuclease variants are preferably prepared by the targeted mutagenesis of the initial meganuclease. The diversity is introduced at positions of the residues contacting the DNA target or interacting (directly or indirectly) with the DNA target. The diversity is preferably introduced in regions interacting with the DNA target, and more preferably introduced at the positions of the interacting amino acids. In libraries generated by targeted mutagenesis, the 20 amino acids can be introduced at the chosen variable positions. Preferably, the amino acids present at the variable positions are the amino acids well-known to be generally involved in protein-DNA interaction.

The custom-made meganuclease is derived from any initial meganuclease. Optionally, the initial meganuclease is selected so as its natural recognition and cleavage site is the closest to the targeted DNA site. Preferably, the initial meganuclease is a homing endonuclease, as specified, in the here above definitions. Homing endonucleases fall into 4 separated families on the basis of well conserved amino acids motifs, namely the LAGLIDADG family, the GIY-YIG family, the His-Cys box family, and the HNH family (Chevalier et al., 2001, N.A.R, 29, 3757-3774).

The detailed three-dimensional structures of several homing endonucleases are known, namely I-Dmo I, PI-Sce I, PI-Pfu I, I-Cre I, I-Ppo I, and a hybrid homing endonuclease I-Dmo I/I-Cre I called E-Dre I (Chevalier et al., 2001, Nat Struct Biol, 8, 312-316; Duan et al., 1997, Cell, 89, 555-564; Heath et al., 1997, Nat Struct Biol, 4, 468-476; Hu et al., 2000, J Biol Chem, 275, 2705-2712; Ichiyanagi et al., 2000, J Mol Biol, 300, 889-901; Jurica et al., 1998, Mol Cell, 2, 469-476; Poland et al., 2000, J Biol Chem, 275, 16408-16413; Silva et al., 1999, J Mol Biol, 286, 1123-1136; Chevalier et al., 2002, Molecular Cell, 10, 895-905).

The LAGLIDADG family is the largest family of proteins clustered by their most general conserved sequence motif: one or two copies of a twelve-residue sequence: the di-dodecapeptide, also called LAGLIDADG motif. The initial LAGLIDADG homing endonuclease can be selected from the group consisting of: I-See I, I-Chu I, I-Dmo I, I-Cre I, I-Csm I, PI-Sce I, PI-Tli I, PI-Mtu I, I-Ceu I, I-Sce II, I-Sce III, HO, PI-Civ I, PI-Ctr I, PI-Aae I, PI-Bsu I, PI-Dha I, PI-Dra I, PI-May I, PI-Mch I, PI-Mfu I, PI-Mfl I, PI-Mga I, PI-Mgo I, PI-Min I, PI-Mka I, PI-Mle I, PI-Mma I, PI-Msh I, PI-Msm I, PI-Mth I, PI-Mtu I, PI-Mxe I, PI-Npu I, PI-Pfu I, PI-Rma I, PI-Spb I, PI-Ssp I, PI-Fac I, PI-Mja I, PI-Pho I, PI-Tag I, PI-Thy I, PI-Tko I, and PI-Tsp I; preferably, I-Sce I, I-Chu I, I-Dmo I, I-Cre I, I-Csm I, PI-Sce I, PI-Pfu I, PI-Tli I, PI-Mtu I, and I-Ceu I; more preferably, I-Dmo I, I-Cre I, PI-Sce I, and PI-Pfu I; still more preferably I-Cre I.

Methods for selecting custom-made meganucleases are well known in the art. Typically, the fragmented approach is preferred for allowing the introduction of a greater diversity. This method is described in document US2006153826 that is herein incorporated by reference.

The meganucleases according to the invention can be used either as a polypeptide or as a polynucleotide construct encoding said polypeptide under the control of appropriate transcription regulatory elements including a promoter, for example a tissue specific and/or inducible promoter. According to an advantageous embodiment of the uses according to the invention, the meganuclease (polypeptide) is associated with:
liposomes, polyethyleneimine (PEI); in such a case said association is administered and therefore introduced into somatic cells target.
membrane translocating peptides (Bonetta, 2002, The Sientist, 16, 38; Ford et al, Gene Ther, 2001, 8, 1-4; Wadia & Dowdy, 2002, Curr Opin Biotechnol, 13, 52-56); in such a case, there is a fusion with said peptides.

Meganucleases can also be introduced into somatic tissue(s) from an individual according to methods generally known in the art which are appropriate for the particular meganuclease and cell type.

According to another advantageous embodiment of the uses according to the invention, the meganuclease (polynucleotide encoding said meganuclease) and/or the targeting DNA is inserted in a vector. Vectors comprising targeting DNA and/or nucleic acid encoding a meganuclease can be introduced into a cell by a variety of methods (e.g., injection, direct uptake, projectile bombardment, liposomes). Meganucleases can be stably or transiently expressed into cells using expression vectors. Techniques of expression in eukaryotic cells are well known to those in the art. (See Current Protocols in Human Genetics: Chapter 12 “Vectors For Gene Therapy” & Chapter 13 “Delivery Systems for Gene Therapy”). Optionally, it may be preferable to incorporate a nuclear localization signal into the recombinant protein to be sure that it is expressed within the nucleus.

Advantageously, the sequence encoding the meganuclease and the targeting DNA are inserted in the same vector.

A vector which can be used in the present invention includes, but is not limited to, a viral vector, a plasmid, a RNA vector or a linear or circular DNA or RNA molecule which may consists of a chromosomal, non chromosomal, semisynthetic or synthetic DNA. Preferred vectors are those capable of autonomous replication (episomal vector) and/or expression of nucleic acids to which they are linked (expression vectors). Large numbers of suitable vectors are known to those of skill in the art and commercially available.

Viral vectors include retrovirus, adenovirus, parvovirus (e.g., adenoassociated viruses), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses such as picornavirus and alphavirus, and double stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, Dtype viruses, HTLV-BLV group, lentivirus, spumavirus (Coffin, J. M., Retroviridae: The viruses and their replication, In Fundamental Virology, Third Edition, B. N. Fields, et al., Eds., Lippincott-Raven Publishers, Philadelphia, 1996). Other examples include murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in McVey et al., U.S. Pat. No. 5,801,030, the teachings of which are incorporated herein by reference.

Vectors can also comprise selectable markers (for example, neomycin phosphotransferase, histidinol dehydrogenase, dihydrofolate reductase, hygromycin phosphotransferase, herpes simplex virus thymidine kinase, adenosine deaminase, glutamine synthetase, and hypoxanthine-guanine phosphoribosyl transferase for eukaryotic cell culture; TRP1 for S. cerevisiae; tetracycline, rifampicin or ampicillin resistance in E. coli; etc. . . .).

Meganucleases and vectors which comprise targeting DNA homologous to the region surrounding the cleavage site and/or nucleic acid encoding a custom-made meganuclease can be introduced into an individual using routes of administration generally known in the art. Administration may be topical or internal, or by any other suitable avenue for introducing a therapeutic agent to a patient. Topical administration may be by application to the skin, or to the eyes, ears, or nose. Internal administration may proceed intradermally, subcutaneously, intramuscularly, intraperitoneally, intraarterially or intravenously, or by any other suitable route. It also may in some cases be advantageous to administer a composition of the invention by oral ingestion, by respiration, rectally, or vaginally.

The meganucleases and vectors can be administered in a pharmaceutically acceptable carrier, such as saline, sterile water, Ringer's solution, and isotonic sodium chloride solution. Typically, for therapeutic applications, the meganucleases will be combined with a pharmaceutically acceptable vehicle appropriate to a planned route of administration. A variety of pharmaceutically acceptable vehicles are well known, from which those that are effective for delivering meganucleases to a site of infection may be selected. The HANDBOOK OF PHARMACEUTICAL EXCIPIENTS published by the American Pharmaceutical Association is one useful guide to appropriate vehicles for use in the invention. A composition is said to be a “pharmaceutically acceptable vehicle” if its administration can be tolerated by the recipient. Sterile phosphate-buffered saline is one example of a pharmaceutically acceptable vehicle that is appropriate for intravenous administration. The mode of administration is preferably at the location of the targeted cells.

The dosage of meganuclease or vector according to the present invention administered to an individual, including frequency of administration, will vary depending upon a variety of factors, including mode and route of administration: size, age, sex, health, body weight and diet of the recipient; nature and extent of symptoms of the disease or disorder being treated; kind of concurrent treatment, frequency of treatment, and the effect desired. For a brief review of pharmaceutical dosage forms and their use, see PHARMACEUTICAL DOSAGE FORMS AND THEIR USE (1985) (Hans Huber Publishers, Berne, Switzerland).

For purposes of therapy, the meganucleases and a pharmaceutically acceptable excipient are administered in a therapeutically effective amount. Such a combination is said to be administered in a “therapeutically effective amount” if the amount administered is physiologically significant. An agent is physiologically significant if its presence results in a detectable change in the physiology of the recipient. In the present context, an agent is physiologically significant if its presence results: in a decrease in the severity of one or more symptoms of the targeted disease, in a genome correction of the lesion or abnormality, or in inhibition of viral infection.

In one embodiment of the uses according to the present invention, the meganuclease is substantially non-immunogenic, i.e., engender little or no adverse immunological response. A variety of methods for ameliorating or eliminating deleterious immunological reactions of this sort can be used in accordance with the invention. In a preferred embodiment, the meganuclease is substantially free of N-formyl methionine. Another way to avoid unwanted immunological reactions is to conjugate meganucleases to polyethylene glycol (“PEG”) or polypropylene glycol (“PPG”) (preferably of 500 to 20,000 daltons average molecular weight (MW)). Conjugation with PEG or PPG, as described by Davis et al., (U.S. Pat. No. 4,179,337) for example, can provide non-immunogenic, physiologically active, water soluble endonuclease conjugates with anti-viral activity. Similar methods also using a polyethylene-polypropylene glycol copolymer are described in Saifer et al. (U.S. Pat. No. 5,006,333).

The meganucleases obtained by the method of the present invention allows rational site directed modifications of RHO gene in a cell which harbours at least one mutation selected from the group consisting of c.263T>C, c.620T>A and c.1031A>C. The purpose of these techniques is to rewrite RHO gene precisely where it should be modified leaving the rest of said gene intact.

Accordingly the present invention further relates to a method for the treatment of retinitis pigmentosa caused by at least one mutation in the RHO gene from the group consisting of c.263T>C, c.620T>A and c.1031A>C comprising the steps consisting of:
1) introducing a double-strand break at the genomic locus in the RHO gene that harbours at least one mutation selected from the group consisting of c.263T>C, c.620T>A and c.1031A>C and that comprises at least one recognition and cleavage site of said meganuclease;
2) maintaining under conditions appropriate for homologous recombination with the chromosomal DNA homologous to the region surrounding the cleavage site.

This strategy is useful to delete a DNA sequence at the target site and therefore impedes the expression of the gene that harbours the mutation.

The invention will be further illustrated by the following figures and examples. However, these examples and figures should not be interpreted in any way as limiting the scope of the present invention.

### EXAMPLE

### Material & Methods

Clinic: Seventy-nine families with a provisional diagnosis of autosomal dominant rod-cone dystrophy, (adRP) were ascertained in the CIC of the Quinze-Vingts hospital, Paris (67 families) and in Montpellier (12 families). Informed consent was obtained from each patient and normal individual controls after explanation of the study and its potential outcome. The study protocol adhered to the tenets of the Declaration of Helsinki and was approved by the local ethics committees. Each patient underwent full ophthalmic examination with assessment of best corrected visual acuity using ETDRS chart, kinetic and static perimetry and colour vision using the desaturated Farnsworth Panel D-15. Full-field and multifocal electroretinography (ERG and mfERG) were performed with DTL recording electrodes and incorporated the ISCEV Standards (Espion² Diagnosys® for full field ERG and Veris II for Multifocal ERG). Severe rod-cone dysfunction was considered when no detectable responses where recorded. Clinical assessment was completed with Fundus Autofluorescence Imaging (FAF) and Optical Coherence Tomography (OCT) (HRAII® and Spectralis® OCT, Heidelberg Engineering, Dossenheim, Germany). At the end of clinical evaluation, patients and family members were asked to donate a blood sample for further genetic studies.

Mutation analysis: Total genomic DNA was extracted from peripheral leucocytes in blood samples by standard salting out procedures or according to manufacturer recommendation (Puregen Kit, Qiagen, Courtaboeuf, France). Subsequently, either genotyping or direct sequencing of RHO was performed. For genotyping 2 to 3 polymorphic microsatellite markers within or contiguous to known adRP genes (RHO, RDS, PRPF31, RP1, PRPF8, IMPDH1, PRPF3, NRL, CA4, CRX, TOPORS, PAP1, NR2E3) was used. Results were analysed with GeneMapper software (version 4.0, Applied Biosystems). The coding 5 exons of rhodopsin (RHO RefSeq NM000539.2) and the flanking intronic regions were amplified with oligonucleotides previously described. At least 125 commercially available control samples were used to validate the pathogenicity of the novel sequence variants (Human random control panel 1-3, Health Protection Agency Culture Collections, Salisbury, United Kingdom).

### RESULTS

Mutation analysis: Thirteen index patients of the investigated 79 French autosomal dominant RP patients revealed a RHO mutation (Table 1). These mutations co-segregated with the phenotype when tested in family members available. Three index patients showed each a novel missense mutation, while ten index patients revealed previously described mutations in RHO (Table 1).
1. Patient CIC00218 from family 155, originating from the Southwest of France, had a novel c.263T>C mutation on exon 1 leading to a p.Leu88Pro substitution
2. Patient CIC00716 of family 475, from Northern France revealed a novel mutation c.620T>A in exon 3 leading to a p.Met207Arg substitution, which segregates with an unusual restricted chorioretinal atrophy phenotype (Audo et al., 2010, in press).
3. Patient CIC00590 from family 394, with Sephardim Jewish origins, revealed a novel mutation, c.1031A>C in exon 5, leading to a p.Gln344Pro substitution.
4. and 5. Two patients from two unrelated families (PB41 and 42) from a similar region in France revealed the known c.44A>G mutation in exon 1 leading to a p.Asn15Ser exchange.
6. Patient CIC00123 from family 172/96 originating from Martinique, within the French West Indies, showed a previously described heterozygous c.392T>C mutation in exon 2 leading to a p.Leu131Pro substitution.
7. and 8. Two index patients CIC00364 and CIC00974 from two unrelated families 247 and 610 respectively revealed the known mutation c.403C>T in exon 2 leading to a p.Arg135Trp substitution, which co-segregated with the disease.
9. Index patient 2296 from family RP827 revealed the earlier described c.998_{—}999insAGGC insertion leading to a predicted frameshift mutation (p.Ser334GlyfsX20), which is assumed to change the open reading frame and elongates the altered protein.
10.-13. Four index patients CIC00161, CIC00841, CIC00944 and CIC01125 from 4 unrelated families with origins in 4 distinct regions of France (family 119, family 546, family 598 and family 681, respectively) revealed the c.1040C>T mutation in exon 5 leading to the p.Pro347Leu substitution, which co-segregated in family members available for genetic testing.

Prevalence of different RHO mutations in France: Together our study on autosomal dominant RP patients from France showed that 16.5% revealed novel or known RHO mutations. Mutations locations revealed no specific hot spots since they involved all exons. However, three mutations occurred at least in two families indicating that the p.Asn15Ser, p.Arg135Trp and the p.Pro347Leu substitutions in RHO are frequent causes of RP in this population.

Phenotypic characteristics of patients with RHO mutation: Thirty affected subjects, between age 8 and 62, from the 13 families found with RHO mutation underwent complete clinical examination. Their phenotypic details are summarized in Table 2. The group of patients reported here shows 3 distinct phenotypes and resemble either class A or B mutants from the classification proposed by Cideciyan and co-workers²¹:
1. a generalized rod-cone dysfunction observed in patients carrying mutations (p.Leu88Pro, p.Leu131Pro, p.Arg135Trp, p.Ser334GlyfsX20, p.Gln344Pro, p.Pro347Leu), which resemble the class A mutants.
2. a sector RP associated with the p. Asn15Ser mutation and
3. a restricted chorioretinal dystrophy predominant at the posterior pole associated with the p.Met207Lys substitution. Due to the more restricted phenotype, we classified the two latter mutations as class B mutations.

In generalized forms, symptoms are classical for RP with no obvious phenotype/genotype differences and are dominated by night blindness, from early childhood, progressive peripheral visual field constriction and late photophobia. Age at time of diagnosis varies from 8 to 49 with a majority within the teenage years, earlier than restricted diseases. Central vision ranges from 20/20 to 20/400. It decreases with age, after peripheral visual field impairment, and is usually relatively conserved up to the 5^{th} decade. However, in 8/21 patients, atrophic changes within the macula occur after the mid-twenties and compromised further central vision. Some degree of cataract or intraocular lens is present as early as 34 in 11/21 patients. Fundus examination, shows in most patients classical RPE changes in the periphery with intraretinal pigment migrations, sign of photoreceptor cell death, increasing with age. White dots are present in 5 patients who are 43 or younger associated with three genotypes are in our series: p.Leu131Pro, p.Gln344Pro and p.Pro347Leu. OCT findings are summarized in Table 2. There was no correlation between OCT abormalities and genotype. Cystoid Macular Edema (CME) is present in 4/30 patients in association with 4 different genotypes. A perifoveal ring of hyper-autofluorescence is present 13/18 patients for whom fundus autofluorescence imaging has been performed. Absence of this ring is associated with irregular loss of autofluorescence within the macula in relation with atrophic changes (FIG. 3). ERG responses are usually undetectable for both scotopic and photopic recordings after 30 or show only residual photopic Flicker responses. When ERGs are detectable, in younger patients, they usually show more decreased amplitudes for scotopic than photopic responses with implicit time shift, consistent with generalized rod-cone dysfunction.

Sector RP was seen in 2 families (PB41 and PB42) carrying the same p.Asn15Ser change. Five patients, from age 28 to 60, underwent full ophthalmic examination. Night blindness is an inconstant sign in these subjects who all retain a normal central vision with inferior peripheral field defect correlated with fundus abnormalities. ERG responses show decreased scotopic responses with additional photopic abnormalities in some patients. There is however no implicit time shift consistent with a restricted rod-cone dysfunction.

One additional family, F475 with a novel p.Met207Arg, shows also restricted chorioretinal degeneration. Phenotype-genotype correlations are described in more details elsewhere (Audo et al., 2010, in press). Briefly, onset of symptoms appears in the fourth decade in this family with moderate night blindness and asymmetric visual loss. Affected family members show patchy areas of chorioretinal atrophy within the posterior pole (FIG. 3) with decreased ERG response amplitudes for both scotopic and photopic responses and no implicit time shift consistent with restricted disease.

### DISCUSSION

The current study reports mutation spectrum on the rhodopsin gene in a cohort of patients from 2 major French centres and further outlines phenotypic variability associated with rhodopsin mutation showing both, generalized or sectorial retinal degeneration. To the best of our knowledge to date only two studies on RHO mutations in a French cohort were published: One describing the prevalence of RHO mutations in Southern France and the other reported on the identification of 5 new mutations with no information on prevalence and ethnic origin (Bareil C, Hamel C, Pallares-Ruiz N, Arnaud B, Demaille J, Claustres M. Molecular analysis of the rhodopsin gene in southern France: identification of the first duplication responsible for retinitis pigmentosa, c.998999ins4. Ophthalmic Genet 1999;20:173-182.; Souied E, Gerber S, Rozet JM, et al. Five novel missense mutations of the rhodopsin gene in autosomal dominant retinitis pigmentosa. Hum Mol Genet 1994;3:1433-1434.).

The overall prevalence of RHO mutations in our cohort is 16.5%. This is consistent with previous reports on European cohorts including Spain (20%), Germany (16%), Italy (16%) and Southern France (10%). This is higher than reports from China (2-7%), Japan (0-6%) India (0-2%) and South-Africa (7%). Studies from the UK and Norway revealed higher numbers with 30-50%. However, the studied cohorts were small (12-20 families) and thus these results must be validated in larger cohorts. In the US population RHO mutations were shown to account for up to 30% of adRP. The prevalence of the p.Pro23His mutation in the US has been reported as high as 12% of adRP due to a founder effect from a common British ancestor. This mutation has never been found in European cohorts of adRP, including the current report, nor in Asian cohorts, which would account for differences in the overall RHO mutation prevalence between the American population and reports from other populations.

Three novel changes were identified in the current study: p.Leu88Pro, p.Met207Lys and p.Gln344Pro.

The p.Leu88Pro substitution leads to a severe generalized rod-cone dystrophy phenotype in the patients. Disease-causing mutations have already been reported for the surrounding residues (namely p.Val87Asp and p.Gly89Asp) and misfolding has been hypothesised as a pathogenic mechanism. The Leucine in 88 is located within the alpha helix of the second transmembrane domain of rhodopsin. The residue at this position is not invariant among Metazoan organism, but shows always hydrophobic characteristics, necessary for the maintenance of this alpha helix. The substitution of the leucine by a proline would induce a kink in the helix and destabilize the protein through rhodopsin misfolding. This would classify the p.Leu88Pro within class II after Mendes and colleagues.

The novel p.Met207Arg substitution was associated with unusual chorioretinal atrophy. Mutation consequences are discussed elsewhere and would suggest a change in sterical constraints within the retinal binding pocket.

The c.1031A>C change in exon 5 leading to a p.Gln344Pro substitution was associated with a severe generalized rod-cone dystrophy. Gln at this position is evolutionary highly conserved. It is located in the C-term external loop and it is unlikely that mutations in this residue would induce a misfolding. This would classify our novel change p.Gln344Pro in class I after Mendes and colleagues. Previously a c.1030C>T change leading to a p.Gln344Stop was associated with normal phototransduction function but with mislocalization. Furthermore, Tai and co-workers identified the direct interaction between a dynein light-chain subunit and the C-terminus of rhodopsin, which is important for the correct protein transport of post-Golgi rhodopsin-containing vesicles along the microtubules up to the outer segment. Different C-terminal mutations were unable to interact with this domain and thus led to a trafficking defect. A similar mechanism can be advocated for the novel reported change p.Gln344Pro.

The 10 other families identified with RHO mutation showed already described changes. The p.Asn15Ser mutation was identified in two different families from a similar region of France and thus represents probably a founder effect. Asn15 represent one of the important N-glycosylation sites of RHO. Thus the underlying pathogenic mechanism of the p.Asn15Ser was proposed to be trafficking defect.

The p.Leu131Pro mutation was identified in a large family from Martinique with typical diffuse rod-cone dystrophy, type A from Cideciyan and co-workers. This amino-acid substitution is assumed to lead to misfolding. Since this exchange has also been previously reported in another study from France, it may represent a major mutation in the affected French population.

The p.Arg135Trp was found in two unrelated families and was associated with typical severe diffuse rod-cone dystrophy, type A from Cideciyan and co-workers as previously reported. Of notes, none of the examined patients in these 2 families demonstrated the white dots previously described in association with this genotype. An explanation would be that the examined patients were either too young or too old to exhibit this distinct feature since, Oh and co-worker have reported the transient nature of these white dots appearing in the second decades of life then fading to leave place to RPE atrophy and bone spicules. It is also noteworthy that these white dots, which are located at the level of the RPE, are not specific of the p.Arg135Trp mutation since it was also seen in association with other RHO mutations in our series and may represent a non-specific sign of photoreceptor degeneration (see table 2 on clinical data).

The p.Pro347Leu mutation was the most prevalent, found in 4 families which would represent 5% of our adRP families. This mutation has also been reported in other populations. Although the 4 families studied herein were unrelated and from different geographical origin, a founder effect cannot be excluded. Haplotype analysis was not performed for this study. However, the gene location is a known hotspot through a higher probability of C>T transition due to a CpG sequence and 6 disease causing amino-acid substitutions have reported at this location (see http://www.retina-international.org/sci-news/rhomut.htm). Again, it was suggested that for these substitutions a trafficking defect represent the pathogenic mechanism. Patients carrying the p.Pro347Leu mutation have a comparable phenotype as patients carrying the p.Gln344Pro and p.Ser334GlnfsX20 changes, all being located at the C-terminus, with early onset-night blindness, and generalized severe rod-cone dystrophy with loss of central vision in the 5^{th} decades. The severity of the disease associated with C-terminal changes within the cytoplasmic domain is well documented in the literature showing a worse prognosis compared in particular to the p.Pro23His mutation located in the N-terminal intradiscal/extracellular portion of the protein. Our cohort in whom genotype-phenotype correlation was performed is still too small to judge the severity associated with a specific mutation but recurrent follow-up will further address this question.

One additional criterion that will need to be further precisely evaluated is the course of macular involvement: perifoveal and foveal atrophy is not uncommon in our series (see table 2 with clinical details) as well as cystoid macular edema which was present in 4/31 patients with no genotype-specificity. These macular changes are responsible for decreased central vision and their prevention should be the major target of future therapeutic interventions.

Further longitudinal studies will precise the course of the disease for each genotype and will help identifying suitable markers and therapeutic windows for photoreceptor rescue, gene replacement or cell based therapies.

TABLE 1
Novel and known RHO mutations in the French cohort.

| | | Nucleotide | | |
|---|---|---|---|---|
| Index (family) | Exon | Exchange | Protein Effect | Publication |
| 2810 (PB41) | 1 | c. 44A>G | p. Asn15Ser | 36 |
| 2923 (PB42) | | | | |
| CIC00218 | 1 | c. 263T>C | p. Leu88Pro | Novel |
| (F155) | | | | |
| CIC00123 | 2 | c. 392T>C | p. Leu131Pro | 26 |
| (F172/96) | | | | |
| CIC00364 | 2 | c. 403C>T | p. Arg135Trp | 4 |
| (F247) | | | | |
| CIC00974 | | | | |
| (F610) | | | | |
| CIC00716 | 3 | c. 620T>A | p. Met207Lys | novel |
| (F475) | | | | (phenotype- |
| | | | | genotype |
| | | | | correlation |
| | | | | published |
| | | | | elsewhere) |
| 2296 (RP827) | 5 | c. | p. | 13 |
| | | 998_999insAGGC | Ser334GlyfsX20 | |
| CIC00590 | 5 | c. 1031A>C | p. G1n344Pro | Novel |
| (F394) | | | | |
| CIC00161 | 5 | c. 1040C>T | p. Pro347Leu | 29 |
| (F119) | | | | |
| CIC00841 | | | | |
| (F546) | | | | |
| CIC00944 | | | | |
| (F598) | | | | |
| CIC01125 | | | | |
| (F681) | | | | |

### REFERENCES

Throughout this application, various references describe the state of the art to which this invention pertains. The disclosures of these references are hereby incorporated by reference into the present disclosure.

1. A method of identifying a subject having or at risk of having or developing a retinitis pigmentosa, comprising detecting in a sample obtained from said subject, the presence of at least one mutation in the rhodopsin (RHO) gene selected from the group consisting of c.263T>C, c.620T>A and c. 1 031A>C wherein the presence of said mutation indicates an increased risk of having or being at risk of having or developing a retinitis pigmentosa.

2. (canceled)

3. A method of treating retinitis pigmentosa in a subject in need thereof, comprising
administering to said subject a therapeutically effective amount of a meganuclease that cleaves a DNA target sequence in a RHO gene locus which comprises a mutation selected from the group consisting of c.263T>C, c.620T>A and c. 1031A>C.
